(19) United States
(12) Patent Application Publication
(10) Pub. No.: US 2024/0173254 A1
(43) Pub. Date: May 30, 2024

(54) TASIPIMIDINE FORMULATIONS AND USE THEREOF

(71) Applicant: ORION CORPORATION, Espoo (FI)

(72) Inventors: Johanna KUJALA, Espoo (FI); Jenni LEHTISALO, Espoo (FI); Jukka SALMIA, Espoo (FI); Kai SINERVO, Espoo (FI)

(21) Appl. No.: 18/551,084

(22) PCT Filed: Mar. 18, 2022
(86) PCT No.: PCT/FI2022/050177

§ 371 Date: Sep. 18, 2023

(30) Foreign Application Priority Data
Mar. 19, 2021 (FI) 20215311

(51) Int. Cl. A61K 9/08; A61K 9/00; A61K 31/4178; A61K 47/12

(52) U.S. Cl. CPC A61K 9/08; A61K 9/0053; A61K 31/4178; A61K 47/12

(57) ABSTRACT

The present disclosure relates to a pharmaceutical composition in the form of an orally deliverable liquid composition comprising tasipimidine, or a pharmaceutically acceptable salt thereof, as an active ingredient and to the use thereof in the treatment and prevention of a disorder, condition or disease where an alpha2A agonist is indicated to be useful, for example, for use as a sedative or analgesic agent, and for use in the treatment of anxiety or agitation. The composition is stable at the pH range from about 2.0 to about 5.0.

### TECHNICAL FIELD

The present disclosure relates to a novel pharmaceutical composition in the form of an orally deliverable liquid pharmaceutical composition comprising tasipimidine, or a pharmaceutically acceptable salt thereof, as an active ingredient and to the use thereof in the treatment and prevention of a disorder, condition or disease where an alpha2A agonist is indicated to be useful, for example, for use as a sedative or analgesic agent, and for use in the treatment of anxiety or agitation.

### BACKGROUND OF THE INVENTION

Alpha2 adrenoceptor agonists have been in clinical use since the mid-1960s when clonidine was introduced as an antihypertensive drug. Alpha2 adrenoceptor activation is known to result in a variety of responses from several organs and tissues. Activation of presynaptic alpha2 adrenoceptors located in sympathetic nerve endings inhibits the release of the neurotransmitter noradrenaline. Activation of postsynaptic alpha2 adrenoceptors in the central nervous system leads to inhibition of sympathetic activity, causing decreases in blood pressure and heart rate, decreased arousal, sedation and relief of anxiety. Activation of alpha2 adrenoceptors at the spinal level results in analgesia. Peripheral alpha2 adrenoceptors in blood vessels mediate vascular smooth muscle contraction. There are three distinct subtypes of alpha2 adrenoceptors, alpha2A, alpha2B and alpha2C, each encoded by their own gene. According to the current knowledge, the major part of the alpha2 adrenergic actions is mediated by the alpha2A subtype.

Currently available, centrally-acting alpha2 agonists are indicated for the treatment of hypertension (clonidine), spasticity (tizanidine), attention deficit hyperactivity disorder (guanfacinc), intensive care sedation and procedural sedation (dexmedetomidine). At sufficiently high dose levels they produce a reduction in blood pressure and heart rate and sedation that are the intended therapeutic effects for some of the compounds, and as adverse effects dry mouth, dizziness, high blood pressure at higher doses, and rarer effects such as atrioventricular conduction block or dissociation particularly in situations with high parasympathetic tone.

Tasipimidine (2-(5-methoxyisochroman-1-yl)-4,5-dihydro-1H-imidazole) is a novel, orally active, highly selective alpha2A adrenoceptor agonist. It is therefore considered that tasipimidine may produce less cardiovascular effects than with older unselective agents. Its high oral bioavailability and alpha2A selectivity differentiate it from dexmedetomidine, the currently approved and most specific alpha2 adrenoceptor agonist. In addition, tasipimidine has a shorter elimination half-life (t1/2) than clonidine, (clonidine t1/2=14 h), faster onset of action, and is more sedative. In dogs tasipimidine has shown to be effective in relieving situational anxiety and fear triggered by noise or owner departure. Tasipimidine is not expected to induce or inhibit enzymes or transporters and is therefore not expected to cause significant interactions with other drugs.

Tasipimidine and pharmaceutically acceptable salts thereof have been disclosed in WO 2013/150173. In addition to indications mentioned above for alpha2 agonists WO 2013/150173 lists other potential indications for tasipimidine, like delirium, hyperactive delirium, insomnia, benzodiazepine or alcohol or opioid or tobacco withdrawal, premature ejaculation, tachycardia, restless leg syndrome, hot flashes, post traumatic stress disorder, pain, chronic pelvic pain syndrome, and breakthrough cancer pain. Tasipimidine and salts thereof, particularly sulfate salt, may be prepared using the method described, for example, in WO 2019/106238.

Alzheimer's disease (AD) is a global public health concern especially in communities with an aging population. The hallmark of AD is progressive cognitive and functional decline. In addition, neuropsychiatric symptoms (NPS) associated with AD are common, severe, and distressing problems that increase caregiver burden, lead to premature institutionalization, and increase the cost of dementia. Agitation and aggression are partly overlapping symptoms that are among the most serious of the NPS associated with AD. These symptoms are potentially dangerous to the patient and others in their environment, and contribute significantly to caregiver stress. They are associated with poor prognosis, more rapid decline of cognitive symptoms, earlier institutionalization, poor quality of life and increased cost of care. Thus, the management of agitation and aggression is a major medical need in the treatment of AD.

Currently, options for the treatment of agitation and aggression remain limited. Treatment guidelines usually recommend environmental and behavioral approaches, prior to considering pharmacotherapy. The medication classes that have been studied include antipsychotics, antidepressants, anticonvulsants, and benzodiazepines. However, the efficacy of these interventions has been modest at best and their use is associated with significant safety issues that are particularly relevant in elderly subjects. The best studied pharmacological interventions for agitation and aggression in AD are the antipsychotics. In meta-analyses, they have demonstrated usually detectable benefits, but their use is associated with increased risk of cerebrovascular adverse events and mortality. Furthermore, several of these compounds carry the risk for extrapyramidal, metabolic, cognitive, and sometimes cardiac QT time related adverse effects. For other medication classes, there are far fewer studies available. Consequently, risperidone and haloperidol are the only compounds approved under specific conditions for short-term use in European countries and no pharmacological agents have regulatory approval for treatment of agitation/aggression in USA. Therefore, safer and more effective pharmacotherapies are clearly required.

The use of alpha2 adrenoceptor agonists in the treatment of agitation and aggression symptoms in AD patients has been suggested due to the central sympatholytic effect of alpha2 adrenoceptor agonists that is alleviating sympathetic (flight and fight) reaction activated during an aggression episode. Experimental support to this has recently emerged from public announcement of successful phase 3 efficacy studies with another alpha2 adrenoceptor agonist dexmedetomidine sublingual film in treatment of acute agitation episodes in both schizophrenia and bipolar disorder patients. In addition, this product has demonstrated efficacy also in a small population of dementia patients with acute agitation. It is assumed that therapeutic effects of tasipimidine on agitation are achieved with dose levels that are borderline sedative but without major impact on orthostatic hypotension and falls in the target population.

WO 2018/126182 describes the use of sublingual dexmedetomidine for the treatment of agitation and WO 2020/006092 describes a specific film formulation containing dexmedetomidine suitable for sublingual administration. WO 2020/006119 describes treatment of agitation by administering dexmedetomidine hydrochloride by intravenous route. In addition, WO 2016/061413 describes the sublingual formulation of dexmedetomidine for the treatment of sleep disorders.

### SUMMARY OF THE INVENTION

It has now been found that tasipimidine, or a pharmaceutically acceptable salt thereof, particularly in the form of oral liquid pharmaceutical composition, is effective medicament for treating a disorder, condition or disease where an alpha2A agonist is indicated to be useful, for example, for use as a sedative or analgesic agent, and for use in the treatment of anxiety or agitation. Said oral liquid pharmaceutical composition, comprises tasipimidine, or a pharmaceutically acceptable salt thereof, at a concentration of at least 0.04 mg/ml, preferably at least 0.20 mg/ml, more preferably at least 0.25 mg/ml. Tasipimidine sulfate drug substance as such has an excellent stability but addition of excipients impair its stability. However, it was found that the present composition is surprisingly stable at the pH range from about 2.0 to about 5.0. Thus, the composition of the present disclosure is particularly suitable for oral delivery in humans. Due to tasipimidine's high alpha2A selectivity, the composition has rapid onset of action without producing significant cardiovascular effects. The therapeutic effects of tasipimidine on e.g. agitation are achieved with dose levels that are borderline sedative but without major impact on orthostatic hypotension. Tasipimidine's high oral bioavailability allows precise and easy administration as oral solution, which is a convenient dosage form for, for example elderly population.

The foregoing as well as other feature and advantages of the present teachings will be more fully understood from the following description and claims.

### DETAILED DESCRIPTION OF THE INVENTION

The present disclosure relates to a novel liquid pharmaceutical composition adapted for oral administration comprising
a) tasipimidine, or a pharmaceutically acceptable salt thereof, as an active ingredient at a concentration of at least 0.04 mg/ml, preferably at least 0.20 mg/ml, more preferably at least 0.25 mg/ml;
b) a buffering agent;
c) a preservative; and
d) water:
wherein the pH of the composition is from about 2.0 to about 5.0, preferably from about 2.1 to 4.0, more preferably from about 2.5 to about 3.5, still more preferably from about 2.9 to about 3.1.

In one embodiment, tasipimidine, or a pharmaceutically acceptable salt thereof, particularly sulfate salt, is used as an active ingredient.

In one embodiment the present disclosure relates to above composition, which is a liquid pharmaceutical composition adapted for oral administration to a mammal, particularly human. The composition is, in particular, adapted for patient self-administration or to be given by their lay or professional caregivers. The composition is particularly useful for the treatment or prevention of anxiety or agitation, and for use as a sedative or analgesic agent, and other diseases were alpha2A agonism is desired.

The actual amount of tasipimidine, or a pharmaceutically acceptable salt thereof, to be administered may depend on numerous factors, such as the sex, age, weight and an overall health status of the subject to be treated, and the particular condition being treated. The amount of composition to be administered in suitably selected such as to provide sufficient anxiety or agitation alleviating effect without inducing significant sedation and/or without causing clinically meaningful effects on blood pressure and/or heat rate in the treated subject. Accordingly, for the treatment or prevention of anxiety or agitation in humans, tasipimidine, or a pharmaceutically acceptable salt thereof, is administered generally in amount of about 0.01-2 mg, preferably about 0.02-1 mg, more preferably about 0.05-0.5 mg, and typically about 0.1-0.2 mg, for example about 0.15 mg. The amount of tasipimidine, or a pharmaceutically acceptable salt thereof, is expressed throughout this document as free base unless otherwise noted. Each amount may be administered to the subject one or multiple times per day.

In one embodiment the present disclosure relates to a composition comprising tasipimidine, or a pharmaceutically acceptable salt thereof, as a sole active ingredient.

In one embodiment the present disclosure relates to the above composition which may comprise in addition to tasipimidine, or a pharmaceutically acceptable salt thereof, one or more other active ingredient(s), particularly those useful in the treatment or prevention of anxiety or agitation in humans.

The composition according to the present disclosure is preferably in the form of an aqueous solution adapted for oral administration to a mammal, particularly human. The concentration of tasipimidine, or a pharmaceutically acceptable salt thereof, should be high enough such that no impractically high amount of solution needs to be administered orally. Thus, the concentration of tasipimidine, or a pharmaceutically acceptable salt thereof, in the aqueous solution composition is at least 0.04 mg/ml, preferably at least 0.20 mg/ml, more preferably at least 0.25 mg/ml. For example, the concentration of tasipimidine, or a pharmaceutically acceptable salt thereof, is generally within the range from about 0.04 mg/ml to 3.0 mg/ml, preferably from about 0.1 mg/ml to 1.0 mg/ml, more preferably from about 0.2 mg/ml to 0.5 mg/ml, for example about 0.3 mg/ml.

It was found that stability of tasipimidine, or a pharmaceutically acceptable salt thereof, is improved in the composition having lower pH value. However, the formulations for oral administration should not have a pH below about 2 so that possible adverse effects like diarrhea, vomiting, tissue ulceration or necrosis and pain on administration could be avoided. The pH of the composition is suitably in the range from about 2.0 to about 5.0, preferably from about 2.1 to about 4.0, more preferably from about 2.5 to about 3.5, still more preferably from about 2.9 to about 3.1, for example about 3.0. At this pH range tasipimidine, or a pharmaceutically acceptable salt thereof, is found to be stable in the composition of the present disclosure. The pH of the composition can be adjusted to the desired range, for example, by using a pH adjusting agent(s). A pH adjusting agent may be a simple acid or base which does not have a pH buffering ability by itself, e.g. HCl or NaOH. Preferably, the solution is buffered. Suitable buffering agents include, but are not limited to, for example, lactic acid/lactate, citric acid/citrate, malic acid/malate, malonic acid/malonate, or phosphoric acid/phosphate buffers. Suitable buffer concentration is about 0.005-3 M, preferably about 0.005-1 M, more preferably about 0.01-1 M, even more preferably about 0.03-0.2 M, for example about 0.1 M. The buffers should be selected so that they do not have any negative effect on the palatability of the formulation. Particularly preferred buffering agent is 0.1 M citric acid/citrate buffer.

The composition suitably also comprise a preservative to inhibit microbial and/or fungal growth in the solution. The preservative is selected from agents that are physicochemically stable and active in the required pH range, do not have any negative effect on the palatability of the formulation and are compatible with the other components of the formulation. Examples of preservatives include, but are not limited to, benzoic acid and salts thereof such as sodium benzoate or potassium benzoate, sorbic acid and salts thereof such as potassium sorbate. Preservatives are commonly used in an amount of about 0.01-1%, preferably about 0.02-0.5%, for example about 0.04-0.2%, per weight of the composition. It was found that benzoic acid salts, such as sodium benzoate are particularly preferred preservatives. Benzoic acid salts, such as sodium benzoate are preferably used in an amount of about 0.02-0.1% per weight of the composition.

The composition may further comprise one or more coloring agent(s). For example, coloring agent can be used to increase the aesthetic appearance or to impart a distinctive appearance, which helps to identify a product in its manufacturing and distribution stages. Or if the colored liquid solution is discharged from the mouth of the patient it can be easily noted.

The composition may further comprise one or more flavoring agent(s). The flavoring agents include, but are not limited to, sweetening agents, artificial flavors, natural flavors, refreshing agents and taste-masking agents, or a combination thereof. The flavoring agent is suitably selected such that it improves patient compliance or the palatability of the solution to humans. In order to maintain the composition in the form of solution, the flavoring agent should also be water soluble, stable and compatible with the other components of the composition. Flavoring agents are generally used in amount of about 0.001-10%, preferably about 0.002-5%, more preferably about 0.002-1%, per weight of the composition.

In one embodiment the present disclosure relates to a liquid pharmaceutical composition adapted for oral administration to mammals, particularly humans, comprising
a) tasipimidine, or a pharmaceutically acceptable salt thereof, as an active ingredient at a concentration of at least 0.04 mg/ml, preferably at least 0.2 mg/ml, more preferably at least 0.25 mg/ml;
b) citric acid/sodium citrate buffer
c) sodium benzoate; and
d) water;
wherein the pH of the composition is from about 2.0 to about 5.0, preferably from about 2.1 to about 4.0, more preferably from about 2.5 to about 3.5, still more preferably from about 2.9 to about 3.1.

In one embodiment the present disclosure relates to a liquid pharmaceutical composition adapted for oral administration to mammals, particularly humans, comprising
a) about 0.004-0.3%, preferably about 0.01-0.1%, more preferably about 0.02-0.05%, per weight of the composition, of tasipimidine or a pharmaceutically acceptable salt thereof;
b) about 0.05-4.5%, preferably about 2.0-2.7%, more preferably about 2.2-2.3%, per weight of the composition, of a buffering agent;
c) about 0.01-1%, preferably about 0.02-0.5%, more preferably about 0.04-0.2%, per weight of the composition, of a preservative; and
d) about 96-98%, preferably about 97-97.9%, more preferably about 97.5-97.8%, per weight of the composition, of water;
wherein the pH of the composition is from about 2.0 to about 5.0, preferably from about 2.1 to about 4.0, more preferably from about 2.5 to about 3.5, still more preferably from about 2.9 to about 3.1.

In one embodiment the present disclosure relates to a liquid pharmaceutical composition adapted for oral administration to mammals, particularly humans, comprising
a) about 0.004-0.3%, preferably about 0.01-0.1%, more preferably about 0.02-0.05%, per weight of the composition, of tasipimidine or a pharmaceutically acceptable salt thereof;
b) about 0.05-4.5%, preferably about 2.0-2.7%, more preferably 2.2-2.3% per weight of the composition, of citric acid/citrate buffer;
c) about 0.01-1%, preferably about 0.02-0.5%, more preferably about 0.04-0.2% per weight of the composition, of a benzoic acid salt; and
d) about 96-98%, preferably about 97-97.9%, more preferably about 97.5-97.8% per weight of the composition, of water;
wherein the pH of the composition is from about 2.0 to about 5.0, preferably from about 2.1 to about 4.0, more preferably from about 2.5 to about 3.5, still more preferably from about 2.9 to about 3.1.

The liquid pharmaceutical composition according to any of the embodiments above is an aqueous solution, i.e. composition where tasipimidine, or a pharmaceutically acceptable salt thereof, is in completely solubilized form.

In one embodiment the present disclosure relates to a method for treating a mammal, particularly human, comprising administering to the subject in need thereof an effective amount of a liquid composition comprising tasipimidine, or a pharmaceutically acceptable salt thereof, as an active ingredient for a time sufficient to give the desired therapeutic effect.

It should be noted that said method for treating a human is intended to encompass all of the potential uses of tasipimidine, including all potential uses which derive from tasipimidine's activity as alpha2A adrenoceptor agonist e.g. its use as hypotensive agent, anxiolytic, analgesic, sedative, and the like. It is especially useful in treating anxiety or agitation or aggression in patients with dementia, e.g. Alzheimer's disease. Agitation may be chronic or acute agitation. It is specifically useful in treating agitation associated with neurodegenerative conditions selected from the group consisting of: Alzheimer disease, frontotemporal dementia, dementia, dementia with Lewy bodies, post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy, senile dementia of the Alzheimer type; or agitation associated with neuropsychiatric conditions selected from the group consisting of: schizophrenia, bipolar disorder, bipolar mania, delirium, and depression, including dementia or mood disorders in subjects with major depression (e.g. stress-related major depression); or agitation associated with other conditions such as OPD/IPD procedures (e.g. MRI, CT or CAT scan, lumbar puncture, bone marrow aspiration/biopsy, tooth extraction and other dental procedures); or agitation associated with alcohol, opioid use disorder, opioid withdrawal and substance abuse withdrawal. Further, it is useful in treating delirium, hyperactive delirium, insomnia, benzodiazepine or alcohol or opioid or tobacco withdrawal, premature ejaculation, tachycardia, restless leg syndrome, hot flashes, post traumatic stress disorder, panic disorder, pain, chronic pelvic pain syndrome, breakthrough cancer pain, traumatic brain injury, and tardive dyskinesia.

In one embodiment the present disclosure relates to a composition comprising tasipimidine, or a pharmaceutically acceptable salt thereof, as an active ingredient for use in the treatment or prevention of anxiety or agitation in humans.

In one embodiment the present disclosure relates to the use of a composition comprising tasipimidine, or a pharmaceutically acceptable salt thereof, as an active ingredient in the manufacture of a medicament for the treatment or prevention of anxiety or agitation in humans.

In one embodiment the present disclosure relates to a composition comprising tasipimidine, or a pharmaceutically acceptable salt thereof, as an active ingredient in the manufacture of a medicament for the treatment or prevention of anxiety or agitation in humans.

In one embodiment the present disclosure relates to a method for the treatment or prevention of anxiety or agitation in humans, comprising administering to the subject in need thereof an effective amount of a composition comprising tasipimidine, or a pharmaceutically acceptable salt thereof, as an active ingredient.

In one embodiment the present disclosure relates to a medicinal kit comprising a) a liquid pharmaceutical composition adapted for oral administration comprising tasipimidine, or a pharmaceutically acceptable salt thereof, as an active ingredient, b) a package for containing said composition, and c) instructions for administering said composition to a mammal, particularly human, for the treatment or prevention of anxiety or agitation. Preferably, said package is a glass bottle and it may further contain an applicator, such as a syringe, capable of dosing a suitable volumes of the composition.

The liquid pharmaceutical composition according to any of the embodiments above can be prepared e.g. by dissolving the active ingredient and excipients to water under stirring, followed by pH adjustment, if necessary.

Pharmaceutically acceptable salts of tasipimidine can be prepared by known methods. Suitable salts include acid addition salts formed, for example, with inorganic acids such as hydrochloric acid, hydrobromic acid, sulfuric acid, nitric acid, phosphoric acid and the like, and organic acids such as acetic acid, fumaric acid, propionic acid, glycolic acid, pyruvic acid, oxalic acid, napthalene-1,5-disulfonic acid, ethane-1,2-disulfonic acid and the like. Sulfate is the preferred salt.

The terms used herein have the meanings indicated below.

The term “tasipimidine”, as used herein refers to 2-(5-methoxyisochroman-1-yl)-4,5-dihydro-1H-imidazole in free form and to pharmaceutically acceptable salts thereof, particularly a sulfate salt.

The term “subject” as used herein, refers to a human patient.

The term “preservative”, as used herein, refers to a compound that inhibits microbial and/or fungal growth in the solution to which it is added.

The term “buffering agent” or “buffer”, as used herein, refers to a compound or combination of compounds that when dissolved in water, resists changes to pH upon addition of acid or base, compared to water without the buffering agent added upon addition of the same amounts of the same acids and bases.

The term “liquid pharmaceutical composition”, as used herein, refers to a pharmaceutical composition comprising a liquid carrier such as water, wherein the active ingredient, such as tasipimidine, or a pharmaceutically acceptable salt thereof, is at least partly, preferably completely, solubilized. Thus, in the preferred embodiment, “liquid pharmaceutical composition” is an aqueous solution.

The term “alleviating”, as used herein, refers to reducing, inhibiting, preventing, suppressing or removing signs of anxiety or agitation.

The present disclosure will be explained in more detailed by the following examples. The examples are meant for illustrating purposes only and do not limit the scope of the invention defined in the claims.

### Example 1: Tasipimidine 0.3 mg/ml Oral Solution Having pH 3.0

| Ingredients | Quantity mg/ml |
|---|---|
| Tasipimidine sulfate | 0.30 (expressed as free base) |
| Trisodium citrate dihydrate | 5.29 |
| Citric acid monohydrate | 17.23 |
| Sodium benzoate | 0.50 |
| Brilliant Blue FCF | 0.01 |
| Tartrazine (E102) | 0.01 |
| Purified water | ad 1.0 ml |

The composition of example 1 was prepared by adding raw materials sequentially into the water and dissolved by mixing.

Following solutions containing 0.3 mg of tasipimidine as a free base and having pH 2-6.9 were prepared according to the method described above.

### Example 2: Solution Having pH 2.0

| Ingredients | Quantity mg/ml |
|---|---|
| Tasipimidine sulfate | 0.30 ((expressed as free base) |
| Sodium citrate dihydrate | — |
| Citric acid monohydrate | 21.01 |
| Sodium benzoate | — |
| Color FD&C blue no 1 | — |
| Color FD&C yellow no 5 | — |
| pH adjustment | — |
| Purified water | ad 1 ml |

### Example 3: Solution Having pH 3.1

| Ingredients | Quantity mg/ml |
|---|---|
| Tasipimidine sulfate | 0.30 (expressed as free base) |
| Sodium citrate dihydrate | 5.29 |
| Citric acid monohydrate | 17.23 |
| Sodium benzoate | 0.50 |
| Color FD&C blue no 1 | 0.01 |
| Color FD&C yellow no 5 | 0.01 |
| pH adjustment | — |
| Purified water | ad 1 ml |

### Example 4: Solution Having pH 3.6

| Ingredients | Quantity mg/ml |
|---|---|
| Tasipimidine sulfate | 0.30 (expressed as free base) |
| Sodium citrate dihydrate | 8.82 |
| Citric acid monohydrate | 14.71 |
| Sodium benzoate | 0.50 |
| Color FD&C blue no 1 | 0.01 |
| Color FD&C yellow no 5 | 0.01 |
| pH adjustment | — |
| Purified water | ad 1 ml |

### Example 5: Solution Having pH 4.1

| Ingredients | Quantity mg/ml |
|---|---|
| Tasipimidine sulfate | 0.30 (expressed as free base) |
| Sodium citrate dihydrate | 12.06 |
| Citric acid monohydrate | 12.40 |
| Sodium benzoate | 0.50 |
| Color FD&C blue no 1 | 0.01 |
| Color FD&C yellow no 5 | 0.01 |
| pH adjustment | — |
| Purified water | ad 1 ml |

### Example 6: Solution Having pH 4.9

| Ingredients | Quantity mg/ml |
|---|---|
| Tasipimidine sulfate | 0.30 (expressed as free base) |
| Sodium citrate dihydrate | 19.12 |
| Citric acid monohydrate | 7.35 |
| Sodium benzoate | — |
| Color FD&C blue no 1 | — |
| Color FD&C yellow no 5 | — |
| pH adjustment | — |
| Purified water | ad 1 ml |

### Example 7: Solution Having pH 5.9

| Ingredients | Quantity mg/ml |
|---|---|
| Tasipimidine sulfate | 0.30 (expressed as free base) |
| Sodium citrate dihydrate | 26.03 |
| Citric acid monohydrate | 2.42 |
| Sodium benzoate | — |
| Color FD&C blue no 1 | — |
| Color FD&C yellow no 5 | — |
| pH adjustment | — |
| Purified water | ad 1 ml |

### Example 8: Solution Having pH 6.9

| Ingredients | Quantity mg/ml |
|---|---|
| Tasipimidine sulfate | 0.30 (expressed as free base) |
| Sodium citrate dihydrate | 29.41 |
| Citric acid monohydrate | — |
| Sodium benzoate | — |
| Color FD&C blue no 1 | — |
| Color FD&C yellow no 5 | — |
| pH adjustment | 0.1M HCl q.s. to pH 7 |
| Purified water | ad 1 ml |

### Experiment 1. Stability Study

An ASAP (Accelerated Stability Assessment Program) study to estimate the stability of tasipimidine sulfate in aqueous solution at 5° C. in pH range of about 2-7 was performed. The solutions of example 2 to 8 were stressed in temperatures 30, 40, 50, 60, 70 and 80° C. for 1-28 days. From the solutions, the main degradation product (N-(2-aminoethyl)-5-methoxy-3,4-dihydro-1H-2-benzopyran-1-carboxamide) was analyzed using HPLC and an estimation of the shelf life at 5° C. using the specification limit of 1.0% for degradation product was calculated. The calculation was performed using ASAP Prime software.

Stress conditions and degradation product results are presented in tables 1 to 7.

TABLE 1
Stress conditions and degradation product results of solution
example 2 (pH 2.0).

| Stress | Stress time | Degradation |
|---|---|---|
| temperature (° C.) | (days) | product (%) |
| 5 | ref | 0.00 |
| 30 | 14 | 0.19 |
| 40 | 14 | 0.60 |
| 50 | 7 | 1.00 |
| 50 | 14 | 1.94 |
| 60 | 2 | 0.87 |
| 60 | 4 | 1.77 |
| 70 | 2 | 2.38 |
| 70 | 4 | 4.60 |
| 80 | 1 | 2.81 |
| 80 | 2 | 5.72 |

TABLE 2
Stress conditions and degradation product results of solution
example 3 (pH 3.1).

| Stress | Stress time | Degradation |
|---|---|---|
| temperature (° C.) | (days) | product (%) |
| 5 | ref | 0.047 |
| 5 | ref | 0.045 |
| 30 | 28 | 0.047 |
| 40 | 14 | 0.045 |
| 40 | 28 | 0.499 |
| 50 | 7 | 0.843 |
| 50 | 14 | 1.637 |
| 60 | 3 | 0.826 |
| 60 | 7 | 1.765 |
| 70 | 2 | 1.530 |
| 70 | 7 | 4.095 |
| 80 | 1 | 2.396 |
| 80 | 2 | 9.189 |

TABLE 3
Stress conditions and degradation product results of solution
example 4 (pH 3.6).

| Stress | Stress time | Degradation |
|---|---|---|
| temperature (° C.) | (days) | product (%) |
| 5 | ref | 0.06 |
| 5 | ref | 0.06 |
| 30 | 28 | 0.62 |
| 40 | 14 | 1.05 |
| 40 | 28 | 2.03 |
| 50 | 7 | 1.05 |
| 50 | 14 | 2.35 |
| 60 | 3 | 1.95 |
| 60 | 7 | 5.00 |
| 70 | 2 | 3.02 |
| 70 | 7 | 10.72 |
| 80 | 1 | 3.70 |
| 80 | 2 | 9.69 |

TABLE 4
Stress conditions and degradation product results of solution
example 5 (pH 4.1).

| Stress | Stress time | Degradation |
|---|---|---|
| temperature (° C.) | (days | product (%) |
| 5 | ref | 0.07 |
| 5 | ref | 0.07 |
| 30 | 28 | 0.80 |
| 40 | 14 | 1.36 |
| 40 | 28 | 2.60 |
| 50 | 7 | 1.46 |
| 50 | 14 | 2.96 |
| 60 | 3 | 2.82 |
| 60 | 7 | 6.93 |
| 70 | 2 | 3.92 |
| 70 | 7 | 14.71 |
| 80 | 1 | 5.34 |
| 80 | 2 | 13.59 |

TABLE 5
Stress conditions and degradation product results of solution
example 6 (pH 4.9).

| Stress | Stress time | Degradation |
|---|---|---|
| temperature (° C.) | (days) | product (%) |
| 5 | ref | 0.00 |
| 30 | 7 | 0.39 |
| 30 | 14 | 0.74 |
| 40 | 4 | 0.73 |
| 40 | 7 | 1.22 |
| 50 | 2 | 1.25 |
| 50 | 4 | 2.39 |
| 60 | 1 | 2.10 |
| 60 | 2 | 4.08 |
| 70 | 1 | 5.98 |
| 70 | 2 | 11.27 |

TABLE 6
Stress conditions and degradation product results of solution
example 7 (pH 5.9).

| Stress | Stress time | Degradation |
|---|---|---|
| temperature (° C.) | (days) | product (%) |
| 5 | ref | 0.09 |
| 30 | 7 | 0.96 |
| 30 | 14 | 1.81 |
| 40 | 4 | 1.95 |
| 40 | 7 | 3.28 |
| 50 | 2 | 3.78 |
| 50 | 4 | 7.24 |
| 60 | 1 | 7.26 |
| 60 | 2 | 14.00 |
| 70 | 1 | 22.50 |
| 70 | 2 | 38.62 |

TABLE 7
Stress conditions and degradation product results of solution
example 8 (pH 6.9).

| Stress | Stress time | Degradation |
|---|---|---|
| temperature (° C.) | (days) | product (%) |
| 5 | ref | 0.29 |
| 30 | 7 | 4.28 |
| 30 | 14 | 7.93 |
| 40 | 4 | 9.87 |
| 40 | 7 | 16.03 |
| 50 | 2 | 20.06 |
| 50 | 4 | 31.63 |
| 60 | 1 | 35.14 |
| 60 | 2 | 41.89 |
| 70 | 1 | 49.93 |
| 70 | 2 | 49.03 |

TABLE 8
ASAP results. Median estimated shelf life and probability to
pass 2 years shelf life for samples with pH from 2.0 to 6.9.

| | Measured solution pH | | | | | | |
|---|---|---|---|---|---|---|---|
| | 2.0 | 3.1 | 3.6 | 4.1 | 4.9 | 5.9 | 6.9 |
| Median estimated | >3.00 | >3.00 | >3.00 | 2.57 | 2.25 | 1.47 | 0.17 |
| shelf life at | | | | | | | |
| 5° C. (years) | | | | | | | |
| Probability to | 100.0 | 100.0 | 99.9 | 87.0 | 60.6 | 28.3 | 0.7 |
| pass 2 years shelf | | | | | | | |
| life at 5° C. (%) | | | | | | | |

The results show clearly that acidic condition protects tasipimidine from degradation. At pH 2.0-3.6, the estimated mean shelf life at 5° C. is >3 years when taking into account the 1.0% specification limit of the self life limiting degradation product.

A person skilled in the art will appreciate that the embodiments described herein can be modified without departing from the inventive concept. A person skilled in the art also understands that the present disclosure is not limited to the particular embodiments disclosed but is intended to also cover modifications of the embodiments that are within the scope of the present disclosure.

1. A liquid pharmaceutical composition comprising:
a) tasipimidine, or a pharmaceutically acceptable salt thereof, as an active ingredient at a concentration of at least 0.04 mg/ml;
b) a buffering agent;
c) a preservative; and
d) water;
wherein the pH of the composition is from about 2.0 to about 5.0; and
wherein the liquid composition is adapted for oral administration.

2. The composition according to claim 1, wherein the active ingredient is tasipimidine sulfate.

3. The composition according to claim 1, wherein the composition is a liquid pharmaceutical composition adapted for oral administration to a human.

4. The composition according to claim 1, wherein the composition comprises about 0.004-0.3%, per weight of the composition, of tasipimidine, or a pharmaceutically acceptable salt thereof.

5. The composition according to claim 1, wherein the buffering agent is citric acid/sodium citrate buffer.

6. The composition according to claim 1, wherein the preservative is a benzoic acid salt.

7. The composition according to claim 6, wherein the preservative is sodium benzoate.

8. The composition according to claim 1, wherein the composition comprises:
a) tasipimidine, or a pharmaceutically acceptable salt thereof, as an active ingredient at a concentration of at least 0.04 mg/ml;
b) citric acid/sodium citrate buffer;
c) sodium benzoate; and
d) water;
wherein the pH of the composition is from about 2.0 to about 5.0.

9. The composition according to claim 1, wherein the composition comprises;
a) 0.004-0.3%, per weight of the composition, of tasipimidine, or a pharmaceutically acceptable salt thereof;
b) 0.05-4.5%, per weight of the composition, of a buffering agent;
c) 0.01-1%, per weight of the composition, of a preservative; and
d) 96-98%, per weight of the composition, of purified water;
wherein the pH of the composition is from about 2.0 to about 5.0.

10. The composition according to claim 9, wherein the composition comprises:
a) 0.01-0.1%, per weight of the composition, of tasipimidine or a pharmaceutically acceptable salt thereof;
b) 2.0-2.7%, per weight of the composition, of citric acid/citrate buffer;
c) 0.02-0.5%, per weight of the composition, of a benzoic acid salt; and
d) 97-97.9%, per weight of the composition, of purified water;
wherein the pH of the composition is from about 2.0 to about 5.0.

11. The composition according to claim 1, wherein the buffer concentration is about 0.005-1 M.

12. (canceled)

13. (canceled)

14. (canceled)

15. (canceled)

16. (canceled)

17. A method for the treatment or prevention of anxiety or agitation in a human, comprising administering to the human subject in need thereof an effective amount of a liquid composition comprising tasipimidine, or a pharmaceutically acceptable salt thereof, as an active ingredient.

18. The method according to claim 17, wherein the composition comprises:
a) tasipimidine, or a pharmaceutically acceptable salt thereof, as an active ingredient at a concentration of at least 0.04 mg/ml;
b) a buffering agent;
c) a preservative; and
d) water:
wherein the pH of the composition is from about 2.0 to about 5.0; and
wherein the liquid composition is adapted for oral administration.

19. The method according to claim 17, wherein the anxiety or agitation is associated with dementia.

20. A medicinal kit comprising a) a liquid pharmaceutical composition adapted for oral administration comprising tasipimidine, or a pharmaceutically acceptable salt thereof, as an active ingredient, b) a package for containing said composition, and c) instructions for administering said composition to a mammal, for the treatment or prevention of anxiety or agitation.

21. The composition according to claim 1 wherein the concentration of tasipimidine, or a pharmaceutically acceptable salt thereof, is at least 0.25 mg/ml.

22. The composition according to claim 1 wherein the pH of the composition is from about 2.9 to 3.1.

23. The composition according claim 1, wherein the composition comprises about 0.02-0.05%, per weight of the composition, of tasipimidine, or a pharmaceutically acceptable salt thereof.

24. The composition according to claim 1, wherein the buffer concentration about 0.1 M.
